(19) United States
(12) Patent Application Publication
(10) Pub. No.: US 2026/0144473 A1
(43) Pub. Date: May 28, 2026

(54) Estimating Blood Pressure Using Single-Lead Electrocardiogram (ECG) Data

(71) Applicant: Bristol-Myers Squibb Company, Princeton, NJ (US)

(72) Inventors: Md Shamsuzzaman, Lawrenceville, NJ (US); Sazedul Alam, Lawrenceville, NJ (US)

(73) Assignee: Bristol-Myers Squibb Company, Princeton, NJ (US)

(21) Appl. No.: 19/395,389

(22) Filed: Nov. 20, 2025

Related U.S. Application Data
(60) Provisional application No. 63/723,740, filed on Nov. 22, 2024.

(51) Int. Cl. A61B 5/346; A61B 5/00; A61B 5/021; A61B 5/256; A61B 5/28

(52) U.S. Cl. CPC A61B 5/346; A61B 5/021; A61B 5/256; A61B 5/28; A61B 5/7207; A61B 5/7225

(57) ABSTRACT

A method includes obtaining single-lead electrocardiogram (ECG) data and processing, using a blood pressure prediction model, the single-lead ECG data to predict a blood pressure of an individual. The blood pressure prediction model is trained by: obtaining a plurality of training samples that each corresponding training single-lead ECG data and a corresponding ground-truth blood pressure; processing, using the blood pressure prediction model, each corresponding training single-lead ECG data to generate a corresponding predicted blood pressure; determining corresponding losses each based on the corresponding predicted blood pressure and the corresponding ground-truth blood pressure; and updating parameters of the blood pressure prediction model based on the corresponding losses.

### CROSS-REFERENCE TO RELATED APPLICATIONS

This U.S. Patent Application claims priority under 35 U.S.C. § 119(e) to U.S. Provisional Application 63/723,740, filed on Nov. 22, 2024. The disclosure of this prior application is considered part of the disclosure of this application and is hereby incorporated by reference in its entirety.

### TECHNICAL FIELD

This disclosure relates to estimating blood pressure using single-lead electrocardiogram (ECG) data.

### BACKGROUND

Cardiovascular disease remains one of the leading causes of mortality worldwide, emphasizing the critical need for efficient and accurate methods of cardiovascular health monitoring. Blood pressure is a vital parameter in assessing cardiovascular health and serves as a key indicator of an individual's overall well-being. Blood pressure is the pressure exerted by blood on the arteries and veins of a patient. Two levels of blood pressure, namely the systolic blood pressure (SBP) and the diastolic blood pressure (DBP), are commonly measured using a cuff-based sphygmomanometer. Both SBP and DBP are critical physiological parameters that may serve as features in various applications, particularly in the fields of healthcare, fitness tracking, and medical research.

### SUMMARY

One aspect of the disclosure provides a computer-implemented method that when executed on data processing hardware causes the data processing hardware to perform operations that include obtaining single-lead electrocardiogram (ECG) data representing electrical activity associated with a heart of an individual, and processing, using a blood pressure prediction model having a convolutional neural network (CNN) model architecture, the single-lead ECG data to predict a blood pressure of the individual. A training process trains the blood pressure prediction model by obtaining training data including a plurality of training samples. Each training sample of the plurality of training samples includes corresponding training single-lead ECG data and a corresponding ground-truth blood pressure. For each training sample of the plurality of training samples, the training process trains the blood pressure prediction model by: processing, using the blood pressure prediction model, the corresponding training single-lead ECG data to generate a corresponding predicted blood pressure; determining a corresponding loss based on the corresponding predicted blood pressure and the corresponding ground-truth blood pressure; and updating parameters of the CNN model based on the corresponding losses.

Implementations of the disclosure may include one or more of the following optional features. In some implementations, obtaining the single-lead ECG data includes obtaining the single-lead ECG data using a single-lead sensor affixed to the individual. In other implementations, obtaining the single-lead ECG data includes obtaining the single-lead ECG data using a single-lead sensor integrated by a wearable device worn by the individual.

The CNN model architecture may include an input layer, one or more one-dimensional convolution layers, a max pooling layer, a flattening layer, and one or more densely-connected layers. A first layer of the one or more one-dimensional convolution layers may include a first plurality of filters, a first rectified linear unit (ReLU) activation function, and a first kernel size, while a second layer of the one or more one-dimensional convolution layers may include a second plurality of filters, a second ReLU activation function, and a second kernel size different from the first kernel size. The max pooling layer may include a size of 2. Moreover, a first layer of the one or more densely-connected layers may include a plurality of nodes and a rectified linear unit (ReLU) activation function.

In some examples, the single-lead ECG data is obtained by pre-processing raw single-lead ECG signals acquired by a single-lead sensor using one or more filters. Here, the one or more filters may be configured to at least one of attenuate noise from a power supply, attenuate high-frequency noise from muscle artifacts, or attenuate low-frequency noise due to baseline wander or breathing motions. Additionally, or alternatively, the one or more filters may include a notch filter to attenuate noise at 50 Hertz (Hz) or 60 Hz, a high-pass filter with a cut-off frequency of 0.05 Hz, a low-pass filter with a cut-off frequency of 150 Hz.

In some implementations, processing, using the blood pressure prediction model having the CNN model architecture, the single-lead ECG data to predict the blood pressure of the individual includes processing, using the blood pressure prediction model having the CNN model architecture, the single-lead ECG data to predict a systolic blood pressure (SBP) value and a diastolic blood pressure (DBP) value of the individual. In these implementations, the corresponding predicted blood pressure generated during the training process may include a corresponding predicted SBP value and a corresponding predicted DBP value, the corresponding ground-truth blood pressure of each training sample may include a corresponding ground-truth SBP value and a corresponding ground-truth DBP value, and determining the corresponding loss may include: determining a corresponding SBP loss based on the corresponding predicted SBP value and the corresponding ground-truth SBP value; and determining a corresponding DBP loss based on the corresponding predicted DBP value and the corresponding ground-truth DBP value.

Another aspect of the disclosure provides a system that includes data processing hardware and memory hardware storing instructions that when executed on the data processing hardware causes the data processing hardware to perform operations. The operations include obtaining single-lead electrocardiogram (ECG) data representing electrical activity associated with a heart of an individual, and processing, using a blood pressure prediction model having a convolutional neural network (CNN) model architecture, the single-lead ECG data to predict a blood pressure of the individual. A training process trains the blood pressure prediction model by obtaining training data including a plurality of training samples. Each training sample of the plurality of training samples includes corresponding training single-lead ECG data and a corresponding ground-truth blood pressure. For each training sample of the plurality of training samples, the training process trains the blood pressure prediction model by: processing, using the blood pressure prediction model, the corresponding training single-lead ECG data to generate a corresponding predicted blood pressure; determining a corresponding loss based on the corresponding predicted blood pressure and the corresponding ground-truth blood pressure; and updating parameters of the CNN model based on the corresponding losses.

This aspect of the disclosure may include one or more of the following optional features. In some implementations, obtaining the single-lead ECG data includes obtaining the single-lead ECG data using a single-lead sensor affixed to the individual. In other implementations, obtaining the single-lead ECG data includes obtaining the single-lead ECG data using a single-lead sensor integrated by a wearable device worn by the individual.

The CNN model architecture may include an input layer, one or more one-dimensional convolution layers, a max pooling layer, a flattening layer, and one or more densely-connected layers. A first layer of the one or more one-dimensional convolution layers may include a first plurality of filters, a first rectified linear unit (ReLU) activation function, and a first kernel size, while a second layer of the one or more one-dimensional convolution layers may include a second plurality of filters, a second ReLU activation function, and a second kernel size different from the first kernel size. The max pooling layer may include a size of 2. Moreover, a first layer of the one or more densely-connected layers may include a plurality of nodes and a rectified linear unit (ReLU) activation function.

In some examples, the single-lead ECG data is obtained by pre-processing raw single-lead ECG signals acquired by a single-lead sensor using one or more filters. Here, the one or more filters may be configured to at least one of attenuate noise from a power supply, attenuate high-frequency noise from muscle artifacts, or attenuate low-frequency noise due to baseline wander or breathing motions. Additionally, or alternatively, the one or more filters may include a notch filter to attenuate noise at 50 Hertz (Hz) or 60 Hz, a high-pass filter with a cut-off frequency of 0.05 Hz, a low-pass filter with a cut-off frequency of 150 Hz.

In some implementations, processing, using the blood pressure prediction model having the CNN model architecture, the single-lead ECG data to predict the blood pressure of the individual includes processing, using the blood pressure prediction model having the CNN model architecture, the single-lead ECG data to predict a systolic blood pressure (SBP) value and a diastolic blood pressure (DBP) value of the individual. In these implementations, the corresponding predicted blood pressure generated during the training process may include a corresponding predicted SBP value and a corresponding predicted DBP value, the corresponding ground-truth blood pressure of each training sample may include a corresponding ground-truth SBP value and a corresponding ground-truth DBP value, and determining the corresponding loss may include: determining a corresponding SBP loss based on the corresponding predicted SBP value and the corresponding ground-truth SBP value; and determining a corresponding DBP loss based on the corresponding predicted DBP value and the corresponding ground-truth DBP value.

The details of one or more implementations of the disclosure are set forth in the accompanying drawings and the description below. Other aspects, features, and advantages will be apparent from the description and drawings, and from the claims.

### DESCRIPTION OF DRAWINGS

FIG. 1 is a schematic view of an example diagnostics system for estimating blood pressure using single-lead electrocardiogram (ECG) data.

FIG. 2 is a schematic view of an example blood pressure estimation machine-learning model.

FIG. 3 is a schematic view of an example training process for training a blood pressure prediction model for estimating blood pressure using single-lead ECG data.

FIG. 4 is an example plot of systolic blood pressure (SBP) and diastolic blood pressure (DBP) values identified in an arterial blood pressure (ABP) signal.

FIG. 5 is a plot of example blood pressure estimation results.

FIG. 6 is a flowchart of an example arrangement of operations for a computer-implemented method of estimating blood pressure using single-lead ECG data.

FIG. 7 is a flowchart of another example arrangement of operations for a computer-implemented method of training the blood pressure prediction model for estimating blood pressure using single-lead ECG data.

FIG. 8 is a schematic view of an example computing device that may be used to implement the systems and methods described herein.

Like reference symbols in the various drawings indicate like elements.

### DETAILED DESCRIPTION

According to The American Heart Association, a patient's blood pressure is considered to be normal when the systolic blood pressure (SBP) is less than 120 mmHg (millimeter of mercury) and the diastolic blood pressure (DBP) is below 80 mmHg. An SBP reading between 120 to 129 and a normal DBP reading less than 80 is considered to be elevated blood pressure. An SBP value between 130 to 139 and a DBP value between 80 to 89 is considered to be Stage 1 hypertension. An SBP value of 140 and above and a DBP pressure value above 90 is classified as Stage 2 hypertension. Hypertension is a major indicator of cardiovascular disease, and if not treated in time, may even lead to death. However, hypertension may remain undiagnosed if the measurement of blood pressure is not carried out in a regular fashion. Further, the blood pressure can vary quite a lot during intra-day activities, especially if one undergoes physical or mental stress. Healthcare providers can use remotely collected blood pressure data to identify deteriorating health conditions and intervene promptly. Traditional techniques of measuring blood pressure often involve cumbersome devices, including cuff-based sphygmomanometers, which are not ideal for continuous monitoring or remote patient care.

As technological advancements progress, the continuous monitoring of blood pressure (continuous blood pressure monitoring) has become increasingly important in the fields of remote patient monitoring and chronic illness management. Continuous blood pressure monitoring enables a more detailed and timely understanding of an individual's blood pressure, which may lead to a proactive and personalized healthcare strategy. Moreover, continuous measurements of blood pressure can be helpful in detecting short-term and long-term blood pressure variability, which is a critical indicator of hypertension. However, traditional blood pressure measurement techniques depend on cuff-based sphygmomanometers, which are not suitable for continuous blood pressure monitoring. Researchers have explored using a combination of electrocardiography (ECG) and photoplethysmography (PPG) sensors in wearable devices to measure SBP and DBP. However, a significant limitation is that many wearable devices do not incorporate both types of sensors. Integrating both an ECG sensor and a PPG sensor into a single wearable device poses challenges, including increased cost, complexity, and size, making it less accessible or comfortable for continuous wear. As a result, many wearables available on the market are equipped with only one of these sensors, typically the PPG sensor, due to its simplicity and lower cost. However, a PPG sensor has significant limitations, underscoring a critical gap in the quest for non-invasive, cuff-less continuous blood pressure monitoring. Therefore, there is a need for reliable, cuff-less, and non-invasive methods for determining blood pressure using a single sensor. For example, obtaining continuous blood pressure measurements using single-lead ECG data captured using a single-lead sensor.

Implementations herein are directed toward training a blood pressure prediction model enabled for continuous prediction of blood pressure values of a patient using only single-lead electrocardiography (ECG) data. By contrast to conventional techniques that predict blood pressure values based on less accurate PPG sensor measurements, cumbersome cuff-based measurement devices, and/or depend on off-line learning frameworks not suitable for continuous blood pressure monitoring, implementations disclosed herein provide the blood pressure prediction model as a deep learning model that is capable of running on wearable devices for continuous prediction of blood pressure values from only single-lead ECG data. For instance, the blood pressure prediction model may be integrated into wearable devices or smartphone/tablet applications to empower individuals and/or their healthcare professionals to monitor their blood pressure in real-time, and thus, promote proactive management of cardiovascular health. By contrast, traditional techniques for blood pressure monitoring are performed off-line, and thus, not suitable for real-time monitoring due to inherent latency issues and server dependencies. Notably, the blood pressure prediction model disclosed herein is developed using deep learning techniques to advantageously provide a non-invasive and continuous monitoring solution for predicting both systolic blood pressure (SBP) and diastolic blood pressure (DBP) values based on single-lead ECG data input to the blood pressure prediction model.

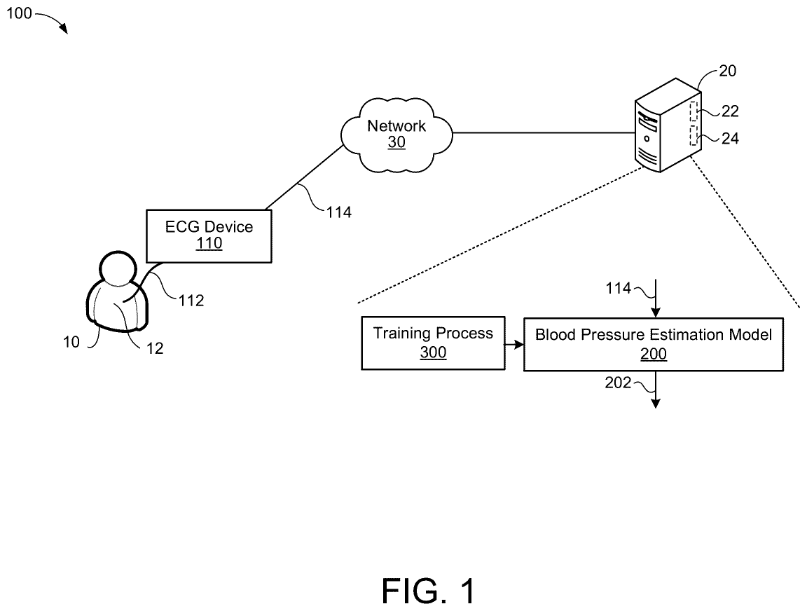

FIG. 1 is a schematic view of a system 100 for training and using a blood pressure prediction model 200 for predicting blood pressure 202 of an individual 10 based on single-lead ECG data 114 (also referred to herein as ECG data 114). The system 100 includes a computing device 20 and the ECG device 110. As used herein, the single-lead ECG data 114 refers to a single-lead ECG waveform or trace recorded by the ECG device 110 from a raw single-lead ECG signal captured by a single-lead sensor 112. As used herein, the single-lead sensor 112 may refer to an ECG electrode configuration that includes a positive electrode and a negative electrode such that a voltage difference therebetween corresponds to the raw single-lead ECG signal. In some configurations, the single-lead sensor 112 additionally includes a third electrode that functions as a reference or ground for reducing noise in the raw single-lead ECG signal. In the example shown, the single-lead sensor 112 includes ECG electrodes affixed to different locations on a chest 12 of the individual 10. In this configuration, the single-lead sensor 112 acquires the raw single-lead ECG signal as the voltage difference between positive and negative ECG electrodes affixed to a corresponding area of the individual's chest 12 representative of electrical activity of their heart. Here, the ECG device 110 may be any type of ECG device including, without limitation, a stationary ECG device, or a portable or mobile ECG device, such as a smart phone or tablet. In some examples, the ECG device 110 includes a wearable device (not shown for clarity of illustration), such as a smartwatch or health monitoring wearable device, that integrates the single-lead sensor 112 at locations to make contact with the individual's skin for acquiring the raw single-lead ECG signal representative of activity of the individual's heart. For example, an ECG device 110 in the form of a smartwatch or specialized wrist worn ECG device 110 may integrate a negative electrode on a back of the device 110 to make continuous contact with the skin of the wrist of the user, while a positive electrode may be integrated on a crown or side of the device 110 such that the individual may touch the positive electrode with a finger from the hand opposite the wrist on which the device 110 is worn to acquire and record the raw single-lead ECG signal.

The computing device 20 (e.g., a computer, a laptop, a tablet, a smartphone, a local server, a remote server, or a server of a distributed system executing in a cloud-computing environment, etc.) is configured to process the single-lead ECG data 114 for estimating a blood pressure 202 of the individual 10 at the time the single-lead ECG data 114 was recorded by the ECG device 110. In the illustrated example, the computing device 20 is in communication with the ECG device 110 via any number and/or type(s) of public and/or private communication network(s) 30 using any number and/or type(s) of wired or wireless digital communication interfaces (e.g., Bluetooth®, USB, etc.). In some examples, the ECG device 110 is a standalone computing device separate from the computing device 20. In other examples, the functionality of the computing device 20 is implemented by the ECG device 110. In some implementations, the computing device 20 receives the single-lead ECG data 114 recorded by the ECG device 110 in real-time as is the raw single-lead ECG signals are acquired by the single-lead sensor 112. Alternatively, the ECG device 110 may store the single-lead ECG data 114 locally and send the ECG data 114 to the computing device 20 periodically or after testing is completed. The computing device 20 includes data processing hardware 22 and memory hardware 24 in communication with the data processing hardware 22. The memory hardware 24 stores instructions that, when executed by the data processing hardware 22, cause the data processing hardware 22 to execute the blood pressure prediction model 200 for predicting the blood pressure 202 of the individual 10 based on the single-lead ECG data 114. As mentioned above, the ECG device 110 may implement the functionality of the computing device 20 such that the ECG device 110 executes the blood pressure prediction model 200 for predicting the blood pressure 202.

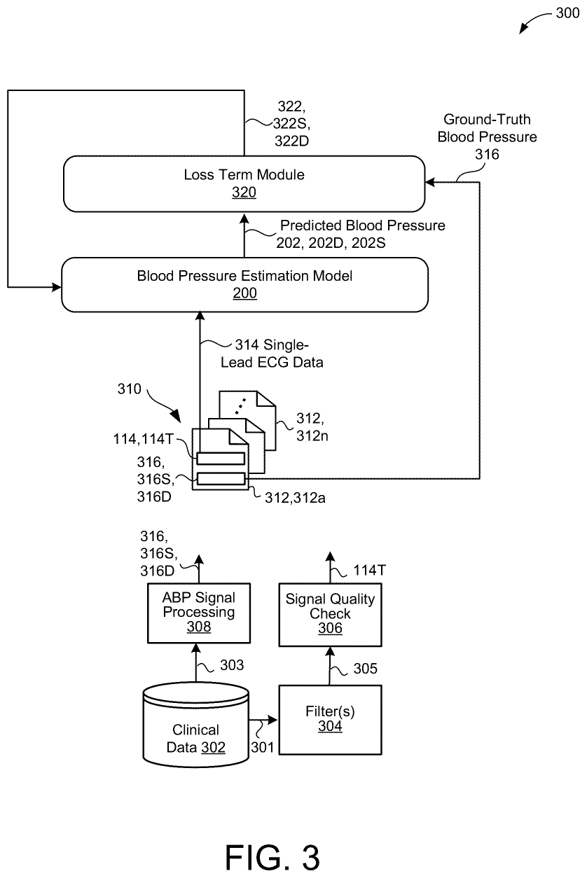

A training process 300 trains the blood pressure prediction model 200 using machine learning techniques. The training process 300 is described in greater detail below with reference to FIG. 3. In some implementations, the blood pressure prediction model 200 includes a convolutional neural network (CNN) model architecture. The blood pressure prediction model 200 may execute on the data processing hardware 22 of the computing device 20 (or the ECG device 110) based on software and/or machine- or computer-readable instructions stored on the memory hardware 24. The blood pressure prediction model 200 processes the single-lead ECG data 114 to predict the blood pressure 202 of the individual 10. In some examples, this process repeats periodically or continuously over time to assess changes in the blood pressure 202 of the individual 10 in a continuous manner over time. In some examples, the ECG data 114 includes streaming data, such that the model 200 continuously or periodically predicts the blood pressure 202. In other examples, the ECG device 110 periodically records ECG data 114 (e.g., every 5 minutes, every 10 minutes, . . . ) based on raw single-lead ECG signals 113 acquired by the single-lead sensor 112 such that the model 200 periodically predicts the blood pressure 202.

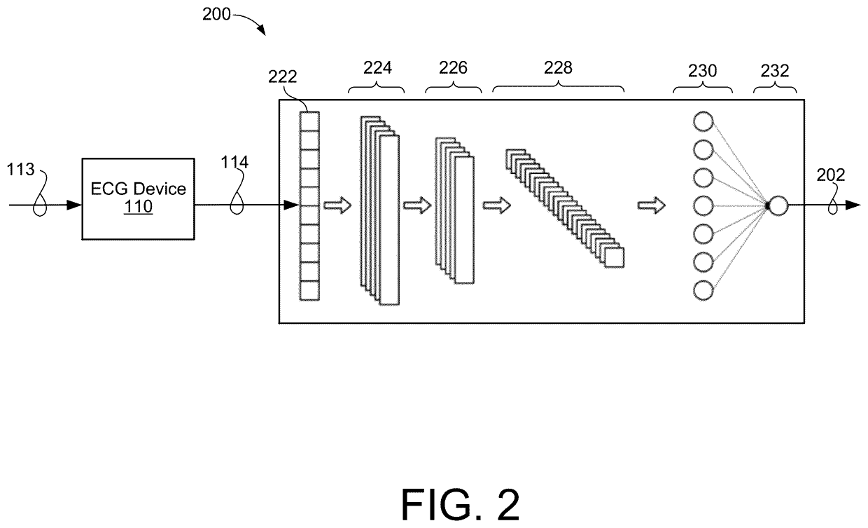

FIG. 2 is a schematic view of an example blood pressure prediction model 200. Initially, the ECG device 110 pre-processes raw single-lead ECG signals 113 acquired by the single-lead sensor 112 using one or more filters to provide smoothed single-lead ECG data 114 as input to the blood pressure prediction model 200. The smoothed single-lead ECG data 114 may correspond to one-dimensional (1D) time-series ECG data 114, such as a single-lead ECG waveform or trace. Here, the one or more filters employed by the ECG device 110 may include filters for attenuating at least one of noise from a power supply, high-frequency noise from muscle artifacts, or low-frequency noise due to baseline wander or breathing motions. Example filters include, but are not limited to, a notch filter to attenuate noise at 50 Hertz (Hz) or 60 Hz, a high-pass filter with a cut-off frequency of 0.05 Hz, and a low-pass filter with a cut-off frequency of 150 Hz. Here, the low-pass filter may include a 10th order Butterworth filter and a Savitzky-Golay filter.

The blood pressure prediction model 200 includes the CNN model architecture having a one-dimensional (1D) input layer 222 configured receive the one-dimensional (1D) time-series ECG data 114 as input. In some examples, the input layer 222 includes 125 samples of the ECG data 114 collected over one second at a sampling frequency is 125 Hz.

The CNN model architecture of the blood pressure prediction model 200 also includes one or more 1D convolutional layers 224 (also referred to as Conv1D layers 224). Here, sequential model initialization initializes the blood pressure prediction model 200 as a stack of linear layers and prepares the CNN model architecture to have layers added thereto. In some examples, the blood pressure prediction model 200 includes a plurality of Conv1D layers 224. Here, the Conv1D layers 224 are one-dimensional suitable for processing the 1D time-series ECG data 114, and the convolutions performed by the Conv1D layers 224 move one-dimensionally only along the time axis. In some examples, a first layer of the Conv1D layers 224 includes sixty-four (64) filters with a kernel size of four (4) and a rectified linear unit (ReLU) activation function, and expects input data with a shape of (n_steps, n_features). In these examples, the n_steps indicates a length of the time sequence and n_features indicates the number of features at each time step. In some additional examples, subsequent layers of the Conv1D layers 224 also include sixty-four filters and a ReLU activation function. However, in some implementations, the Conv1D layers 224 subsequent to the first layer of the Conv1D layers vary in kernel size and, thus, progressively analyze the ECG data 114 at different scales or granularities. The plurality of Conv1D layers 224 may include a first layer that includes a first plurality of filters, a first ReLU activation function, and a first kernel size. The plurality of Conv1D layers 224 may also include a second layer subsequent to the first layer and that includes a second plurality of filters, a second ReLU activation function, and a second kernel size. The second kernel size may be different than the first kernel size.

The CNN model architecture of the blood pressure prediction model 200 also includes a 1D max pooling layer 226 (also referred to as MaxPooling1D layer 226) configured to receive the output of the Conv1D layers 224. The MaxPooling1D layer 226 uses a pool size of two (2). The MaxPooling1D layer 226 reduces the dimensionality of the data by taking the maximum value over the window defined by the pool size, thereby effectively downsampling the output of the Conv1D layers 224 by a factor of two (2). Here, the downsampling helps reduce the number of computations and may help in extracting higher-level features from the local features identified by the Conv1D layers 224.

After the MaxPooling1D layer 226, the CNN model architecture of the blood pressure prediction model 200 includes a flattening layer 228 that transforms the multi-dimensional output of the previous layers into a 1D array for subsequent processing by one or more densely-connected layers 230, 232. In some examples, the one or more densely-connected layers includes a first densely-connected layer 230 and a second densely-connected layer 232. In these examples, the first densely-connected layer 230 may include fifty (50) nodes or neurons and a ReLu activation function configured to further abstract the features into a higher-level representation. In these examples, the second densely-connected layer 232 defines an output layer with a single node or neuron and does not include a ReLu activation function so that the predicted blood pressure 202 output from the second densely-connected layer 232 is directly output as a continuous value.

FIG. 3 is a schematic view of an example training process 300 for training the blood pressure prediction model 200 having the CNN model architecture. The training process 300 may execute on the computing device 20 (i.e., on the data processing hardware 22) or on another computing system, such as a remote physical or virtual server. In the example shown, the training process 300 trains the blood pressure prediction model 200 using a training data set 310 that includes a plurality of training samples 312, 312a-n. Here, each corresponding training sample 312 of the plurality of training samples 312 includes corresponding training single-lead ECG data 114, 114T paired with a corresponding ground-truth blood pressure value 316. The corresponding ground-truth blood pressure value 316 may indicate both a ground-truth SBP value 316S and a ground-truth DBP value 316D for the corresponding training single-lead ECG data 114T.

For each corresponding training sample 312 in the training data set 310, the training process 300 processes, using the blood pressure prediction model 200 having the CNN model architecture, the corresponding training single-lead ECG data 114T to generate a predicted blood pressure value 202. Here, the predicted blood pressure value 202 may indicate both a predicted SBP value 202S and a predicted DBP value 202D for the corresponding training sample 312. Optionally, the predicted blood pressure value 202 may indicate one of the predicted SBP value 202S or the predicted DBP value 202D. A loss term module 320 determines a corresponding loss 322 based on the corresponding ground-truth blood pressure value 316 and the predicted blood pressure value 202 output from the blood pressure prediction model 200. In some examples, the loss term module 320 determines a SBP loss 322S and a DPB loss 322D for each corresponding training sample 312. Here, the SBP loss 322S represents a numerical difference between the corresponding ground-truth SBP value 316S and the corresponding predicted SBP value 202S, while the DPB loss 322D represents a numerical difference between the corresponding ground-truth DBP value 316D and the corresponding predicted DBP value 202D.

Thereafter, the training process 300 trains the model 200 based on the corresponding losses 322 to teach the CNN model 200 to learn how to predict blood pressures 202 based on single-lead ECG data 114. In some examples, the training process 300 trains the blood pressure prediction model 200 by adjusting, adapting, updating, fine-tuning, etc. one or more parameters and/or weights of the CNN model architecture based on the corresponding losses 322.

In the example shown, the training process 300 generates the training data set 310 based on collected clinical data 302. In some examples, the clinical data 302 is based on the Multi-Parameter Intelligent Monitoring in Intensive Care (MIMIC) II online waveform database, which is made available by the PhysioNet organization. Here, the online MIMIC II waveform database includes a multitude of waveform signals that were recorded by patient monitors at several hospitals over a time period spanning from 2001 to 2008. The waveform signals were sampled at a frequency of 125 Hz, with a minimum precision of 8 bits. A data collector program collected raw ECG signals 301, raw arterial blood pressure (ABP) signals 303, and PPG signals from the online waveform database, and then preprocessed and cleaned the collected data.

The training process 300 may apply one or more filters 304 to the raw ECG signals 301 to ensure accurate interpretation and diagnosis. For example, raw ECG signal often contain unwanted noise and interference from various sources, such as mains frequencies and muscle artifacts, which can obscure important cardiac information. To mitigate these issues, the one or more filters 304 of the training process 300 apply a variety of filtering techniques to the raw ECG signals 301. For example, a notch filter may be utilized to specifically target and reduce interference from mains (e.g., from nearby electrical equipment or power supply) frequencies, and a low-pass filter may be utilized to attenuate high-frequency noise such as muscle artifacts. Conversely, a high-pass filter may be utilized to help eliminate low-frequency interference, such as baseline wander caused by breathing motions. By carefully selecting and applying appropriate filters 304, the integrity of the training ECG data 114T can be preserved, enabling clinicians to accurately analyze and diagnose cardiac abnormalities. Example filters 304 include, but are not limited to, a notch filter to attenuate noise at 50 Hertz (Hz) or 60 Hz, a high-pass filter with a cut-off frequency of 0.05 Hz, and a low-pass filter with a cut-off frequency of 150 Hz. Here, the low-pass filter may include a 10th order Butterworth filter and a Savitzky-Golay filter.

The training process 300 may also apply one or more signal quality checks 306 to the filtered ECG data 305 to detect or flag artifacts or irregularities in the filtered ECG data 305. The filtered ECG data 305 includes the raw ECG signal 301 after application of the one or more filters 304. Algorithms, like those found in signal processing software such as MATLAB or Python's BioSPPY library, may be used for detecting and flagging artifacts or irregularities in the filtered ECG data 305. For example, a finite impulse response (FIR) bandpass filter may be used to eliminate low-frequency and high-frequency components from the filtered ECG data 305. Additionally, or alternatively, variability in the intervals between successive heartbeats may be analyzed to determine the quality of the filtered ECG data 305 and/or to detect heartbeats and compute inter-beat intervals (IBIs) representing the time difference between two consecutive heartbeats. A heart rate (HR) may be determined using the IBI values. Thereafter, if the HR value deviated outside the range of typical or expected physiologically values, the corresponding segment of the filtered ECG data 305 is flagged as unusable, indicating potential issues with signal quality. In some examples, HR values between 40 to 240 bpm (beat per minute) are considered to be acceptable for modeling purpose. This meticulous quality check process is important to ensuring the reliability and accuracy of the training ECG data 114T.

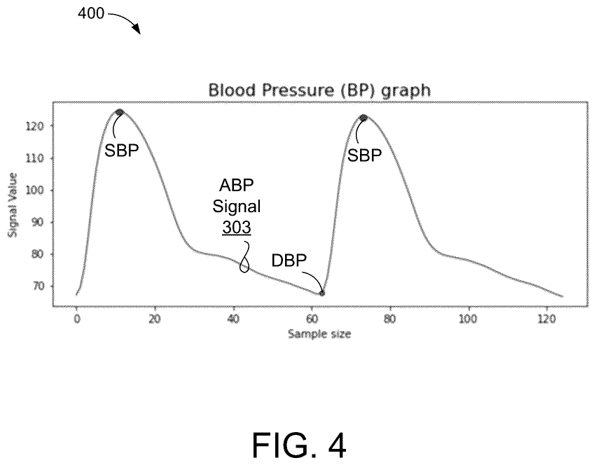

The training process 300 may apply ABP signal processing 308 to the raw APB signals 303 to extract the ground-truth SBP and DBP values 316S, 316D from the ABP signals 303. Here, the ABP signal processing 308 may employ peak and trough detection algorithms to identify the peaks and troughs in the raw ABP signals 303. In some examples, peaks of an ABP signal 303 are identified as ground-truth SBP values 316S, and troughs of the ABP signal 303 are identified as ground-truth DPB values 316D. FIG. 4 is an example plot 400 of an ABP signal 303 showing SBP values identified at the peaks of the ABP signal 303 and a DPB value identified at a trough of the ABP signal 303. The x-axis denotes sample size in Hz and the y-axis denotes blood pressure values in mmHg.

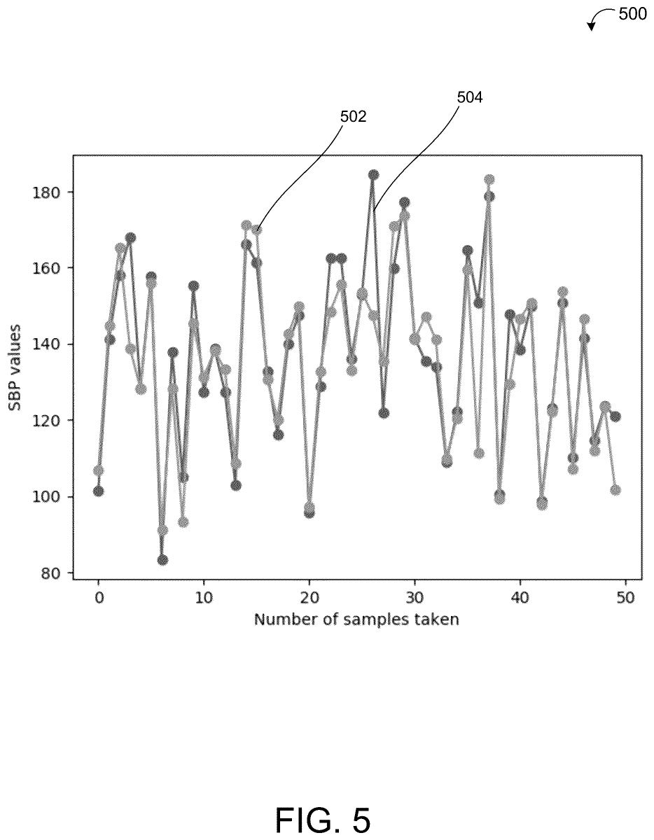

FIG. 5 is a plot 500 of example blood pressure estimation results. The plot 500 includes a first line 502 representing estimated SBP values, and a second line 504 representing measured SBP values. As shown, the model 200 accurately predicts SBP values. Here, the mean absolute error is 6.04, and the mean absolute percentage error is 4.57. The correlation coefficient between predicted SBP values and measured SBP values is 0.87, with a mean difference of −0.96, and a standard deviation of +/−11.2. The error is less than 5 mmHg 43.8% of the time, less than 10 mmHg 71.5% of the time, and less than 15 mmHg 85.5% of the time.

While not shown in the plot 500 of FIG. 5, the trained blood prediction model 200 also accurately predicts DBP values. Here, the mean absolute error is 6.01, and the mean absolute percentage error is 4.54. The correlation coefficient between predicted DBP values and measured DBP values is 0.78, with a mean difference of −0.1 and a standard deviation of +/−7.04. The error is less than 5 mmHg 68.8% of the time, less than 10 mmHg 90.3% of the time, and less than 15 mmHg 96.1% of the time.

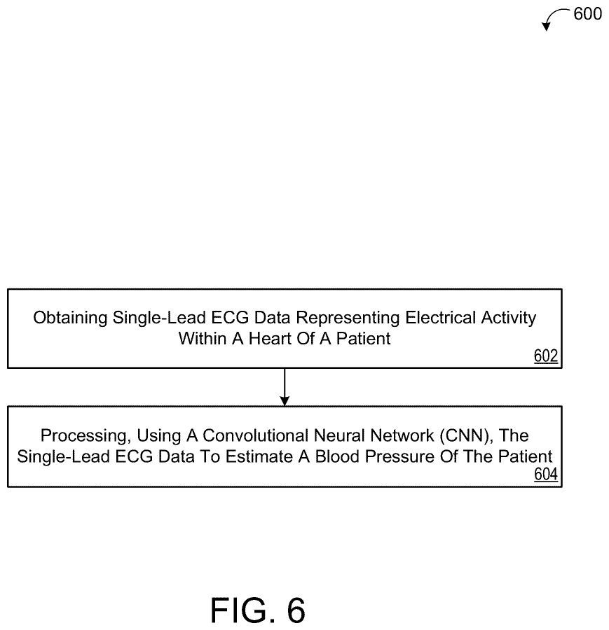

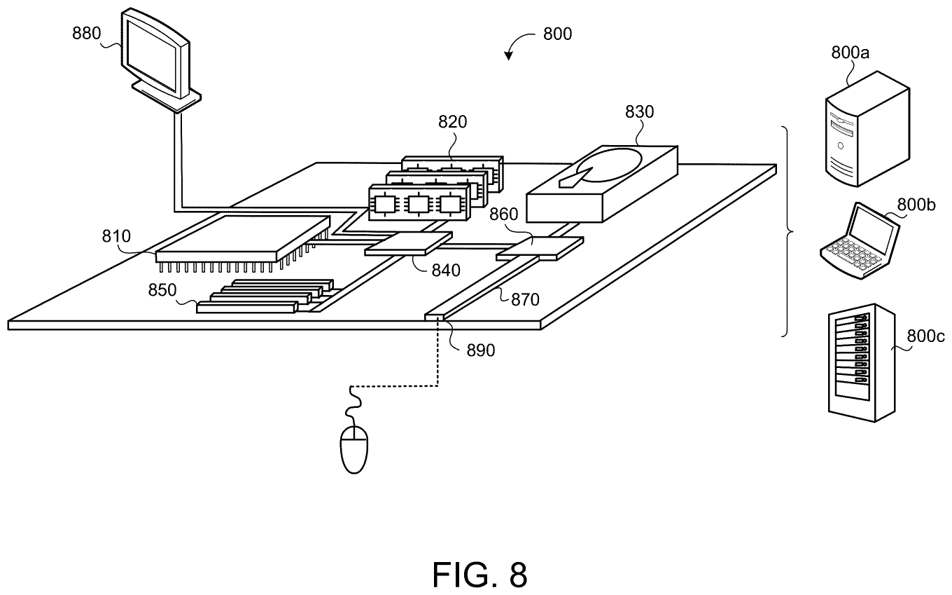

FIG. 6 is a flowchart of an exemplary arrangement of operations for a computer-implemented method 600 of using a blood pressure prediction model 200 for predicting blood pressure 202 of an individual 10 based on single-lead ECG data 114. The computer-implemented method 600 may execute on data processing hardware 810 (FIG. 8) that causes the data processing hardware 810 to perform the operations. The data processing hardware 810 may execute instructions stored on memory hardware 820 (FIG. 8) that causes the data processing hardware 810 to perform the operations. The data processing hardware 810 may include the data processing hardware 22 of the computing device 20 (or the ECG device 110) and the memory hardware 820 may include the memory hardware 24 of the computing device 20 (or the ECG device 110).

At operation 602, the method 600 includes obtaining the single-lead ECG data 114 representing electrical activity associated with the heart of the individual 10. At operation 604, the method 600 includes processing, using the blood pressure prediction model 200 having the CNN model architecture, the single-lead ECG data 114 to generate the predicted blood pressure 202 of the individual 10 processing, using a blood pressure prediction model having a convolutional neural network (CNN) model architecture, the single-lead ECG data to predict a blood pressure of the individual. The predicted blood pressure 202 may include both an estimated/predicted SBP value and an estimated/predicted DPB value. Optionally, the blood pressure prediction model 200 having the CNN model architecture may be trained to only predict/estimate only one of the SBP value or the DPB value.

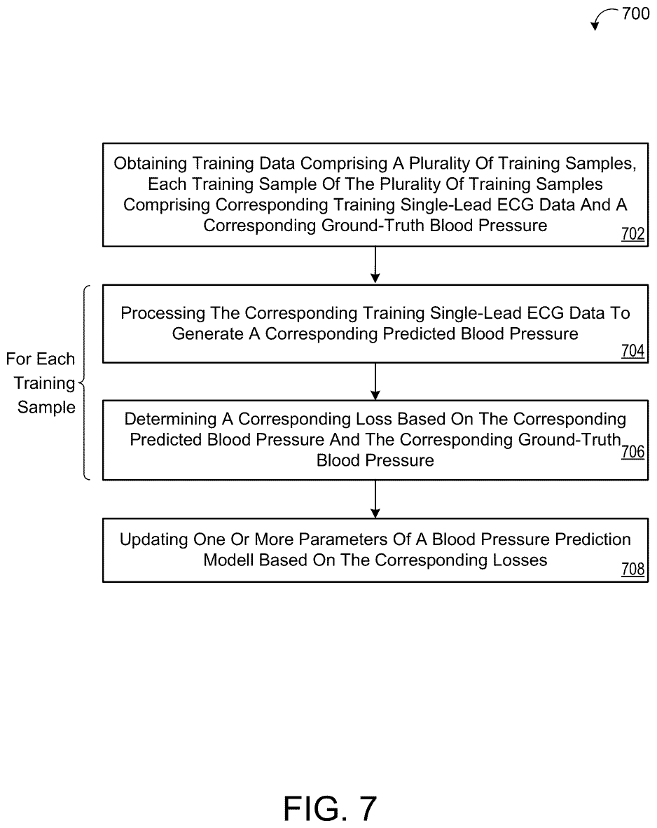

FIG. 7 is a flowchart of an exemplary arrangement of operations for a computer-implemented method 700 of training a blood pressure prediction model 200 to learn how to generate a predicted blood pressure 202 based on single-lead ECG data 114. The flowchart of FIG. 7 may be described with reference to the training process 300 of FIG. 3. The blood pressure prediction model 200 includes a CNN model architecture. Notably, the blood pressure prediction model 200 may be trained to predict/estimate both SBP and DPB values 202 from the single-lead ECG data 114. The computer-implemented method 700 may execute on data processing hardware 810 (FIG. 8) that causes the data processing hardware 810 to perform the operations. The data processing hardware 810 may execute instructions stored on memory hardware 820 (FIG. 8) that causes the data processing hardware 810 to perform the operations. The data processing hardware 810 may include the data processing hardware 22 of the computing device 20 and the memory hardware 820 may include the memory hardware 24 of the computing device 20. The computer-implemented method 700 for performing the operations associated with the training process 300 may be executed on a remote computing system different than the computing device 20 and the ECG device 110. That is, after the training process 300 trains the blood pressure prediction model 200 on the remote computing system, the trained blood pressure prediction model 200 having the CNN model architecture may be loaded and/or accessed by any number of computing devices 20 and/or ECG devices 110 for continuous blood pressure monitoring of individuals.

At operation 702, the method 700 includes obtaining a training data set 310 that includes a plurality of training samples 312 each including corresponding training single-lead ECG data 114, 114T paired with a corresponding ground-truth blood pressure value 316. The corresponding ground-truth blood pressure value 316 may indicate both a ground-truth SBP value 316S and a ground-truth DBP value 316D for the corresponding training single-lead ECG data 314.

At operation 704, for each training sample 312 of the plurality of training samples 312, the method 700 includes processing, using the blood pressure prediction model 200 having the CNN model architecture, the corresponding training single-lead ECG data 114T to generate a corresponding predicted blood pressure value 202. Here, the corresponding predicted blood pressure value 202 may indicate both a corresponding predicted SBP value 202S and a corresponding predicted DBP value 202D for the corresponding training sample 312.

At operation 706, for each training sample 312 of the plurality of training samples 312, the method 700 includes determining a corresponding loss 322 based on the corresponding ground-truth blood pressure value 316 and the predicted blood pressure value 202. In some examples, determining the corresponding loss 322 includes determining a SBP loss 322S and a DPB loss 322D for each corresponding training sample 312. Here, the SBP loss 322S represents a numerical difference between the corresponding ground-truth SBP value 316S and the corresponding predicted SBP value 202S, while the DPB loss 322D represents a numerical difference between the corresponding ground-truth DBP value 316D and the corresponding predicted DBP value 202D. At operation 708, the method 700 includes updating one or more parameters of the blood pressure prediction model 220 based on the corresponding losses 322.

FIG. 8 is a schematic view of an example computing device 800 that may be used to implement the systems and methods described in this document. The computing device 800 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 800 includes a processor 810 (i.e., data processing hardware) that can be used to implement the data processing hardware 22, memory 820 (i.e., memory hardware) that can be used to implement the memory hardware 24, a storage device 830 (i.e., memory hardware) that can be used to implement the memory hardware 24 or the training data set 310, a high-speed interface/controller 840 connecting to the memory 820 and high-speed expansion ports 850, and a low speed interface/controller 860 connecting to a low speed bus 870 and a storage device 830. Each of the components 810, 820, 830, 840, 850, and 860, are interconnected using various busses, and may be mounted on a common motherboard or in other manners as appropriate. The processor 810 can process instructions for execution within the computing device 800, including instructions stored in the memory 820 or on the storage device 830 to display graphical information for a graphical user interface (GUI) on an external input/output device, such as display 880 coupled to high-speed interface 840. In other implementations, multiple processors and/or multiple buses may be used, as appropriate, along with multiple memories and types of memory. Also, multiple computing devices 800 may be connected, with each device providing portions of the necessary operations (e.g., as a server bank, a group of blade servers, or a multi-processor system).

The memory 820 stores information non-transitorily within the computing device 800. The memory 820 may be a computer-readable medium, a volatile memory unit(s), or non-volatile memory unit(s). The non-transitory memory 820 may be physical devices used to store programs (e.g., sequences of instructions) or data (e.g., program state information) on a temporary or permanent basis for use by the computing device 800. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random-access memory (DRAM), static random-access memory (SRAM), phase change memory (PCM) as well as disks or tapes.

The storage device 830 is capable of providing mass storage for the computing device 800. In some implementations, the storage device 830 is a computer-readable medium. In various different implementations, the storage device 830 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 820, the storage device 830, or memory on processor 810.

The high-speed controller 840 manages bandwidth-intensive operations for the computing device 800, while the low-speed controller 860 manages lower bandwidth-intensive operations. Such allocation of duties is exemplary only. In some implementations, the high-speed controller 840 is coupled to the memory 820, the display 880 (e.g., through a graphics processor or accelerator), and to the high-speed expansion ports 850, which may accept various expansion cards (not shown). In some implementations, the low-speed controller 860 is coupled to the storage device 830 and a low-speed expansion port 890. The low-speed expansion port 890, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 800 may be implemented in a number of different forms, as shown in the figure. For example, it may be implemented as a standard server 800a or multiple times in a group of such servers 800a, as a laptop computer 800b, or as part of a rack server system 800c.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

These computer programs (also known as programs, software, software applications, or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random-access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Unless expressly stated to the contrary, the phrase “at least one of A, B, or C” is intended to refer to any combination or subset of A, B, C such as: (1) at least one A alone; (2) at least one B alone; (3) at least one C alone; (4) at least one A with at least one B; (5) at least one A with at least one C; (6) at least one B with at least C; and (7) at least one A with at least one B and at least one C. Moreover, unless expressly stated to the contrary, the phrase “at least one of A, B, and C” is intended to refer to any combination or subset of A, B, C such as: (1) at least one A alone; (2) at least one B alone; (3) at least one C alone; (4) at least one A with at least one B; (5) at least one A with at least one C; (6) at least one B with at least one C; and (7) at least one A with at least one B and at least one C. Furthermore, unless expressly stated to the contrary, “A or B” is intended to refer to any combination of A and B, such as: (1) A alone; (2) B alone; and (3) A and B.

A number of implementations have been described. Nevertheless, it will be understood that various modifications may be made without departing from the spirit and scope of the disclosure. Accordingly, other implementations are within the scope of the following claims.

What is claimed is:

1. A computer-implemented method executed on data processing hardware that causes the data processing hardware to perform operations comprising:
obtaining single-lead electrocardiogram (ECG) data representing electrical activity associated with a heart of an individual; and
processing, using a blood pressure prediction model having a convolutional neural network (CNN) model architecture, the single-lead ECG data to predict a blood pressure of the individual,
wherein a training process trains the blood pressure prediction model by:
obtaining training data comprising a plurality of training samples, each training sample of the plurality of training samples comprising corresponding training single-lead ECG data and a corresponding ground-truth blood pressure;
for each training sample of the plurality of training samples:
processing, using the blood pressure prediction model, the corresponding training single-lead ECG data to generate a corresponding predicted blood pressure; and
determining a corresponding loss based on the corresponding predicted blood pressure and the corresponding ground-truth blood pressure; and
updating parameters of the blood pressure prediction model based on the corresponding losses.

2. The computer-implemented method of claim 1, wherein obtaining the single-lead ECG data comprises obtaining the single-lead ECG data using a single-lead sensor affixed to the individual.

3. The computer-implemented method of claim 1, wherein obtaining the single-lead ECG data comprises obtaining the single-lead ECG data using a single-lead sensor integrated by a wearable device worn by the individual.

4. The computer-implemented method of claim 1, wherein the CNN model architecture comprises:
an input layer;
one or more one-dimensional convolution layers;
a max pooling layer;
a flattening layer; and
one or more densely-connected layers.

5. The computer-implemented method of claim 4, wherein:
a first layer of the one or more one-dimensional convolution layers comprises a first plurality of filters, a first rectified linear unit (ReLU) activation function, and a first kernel size; and
a second layer of the one or more one-dimensional convolution layers comprises a second plurality of filters, a second ReLU activation function, and a second kernel size different from the first kernel size.

6. The computer-implemented method of claim 4, wherein the max pooling layer comprises a pool size of 2.

7. The computer-implemented method of claim 4, wherein a first layer of the one or more densely-connected layers comprises a plurality of nodes and a rectified linear unit (ReLU) activation function.

8. The computer-implemented method of claim 1, wherein the single-lead ECG data is obtained by pre-processing raw single-lead ECG signals acquired by a single-lead sensor using one or more filters.

9. The computer-implemented method of claim 8, wherein the one or more filters are configured to at least one of:
attenuate noise from a power supply;
attenuate high-frequency noise from muscle artifacts; or
attenuate low-frequency noise due to baseline wander or breathing motions.

10. The computer-implemented method of claim 8, wherein the one or more filters comprise:
a notch filter to attenuate noise at 50 Hertz (Hz) or 60 Hz;
a high-pass filter with a cut-off frequency of 0.05 Hz; and
a low-pass filter with a cut-off frequency of 150 Hz.

11. The computer-implemented method of claim 1, wherein processing, using the blood pressure prediction model having the CNN model architecture, the single-lead ECG data to predict the blood pressure of the individual comprises processing, using the blood pressure prediction model having the CNN model architecture, the single-lead ECG data to predict a systolic blood pressure (SBP) value and a diastolic blood pressure (DBP) value of the individual.

12. The computer-implemented method of claim 11, wherein:
the corresponding predicted blood pressure generated during the training process comprises a corresponding predicted SBP value and a corresponding predicted DBP value;
the corresponding ground-truth blood pressure of each training sample comprises a corresponding ground-truth SBP value and a corresponding ground-truth DBP value; and
determining the corresponding loss comprises:
determining a corresponding SBP loss based on the corresponding predicted SBP value and the corresponding ground-truth SBP value; and
determining a corresponding DBP loss based on the corresponding predicted DBP value and the corresponding ground-truth DBP value.

13. A system comprising:
data processing hardware; and
memory hardware in communication with the data processing hardware, the memory hardware storing instructions that, when executed on the data processing hardware, cause the data processing hardware to perform operations comprising:
obtaining single-lead electrocardiogram (ECG) data representing electrical activity associated with a heart of an individual; and
processing, using a blood pressure prediction model having a convolutional neural network (CNN) model architecture, the single-lead ECG data to predict a blood pressure of the individual,
wherein a training process trains the blood pressure prediction model by:
obtaining training data comprising a plurality of training samples, each training sample of the plurality of training samples comprising corresponding training single-lead ECG data and a corresponding ground-truth blood pressure;
for each training sample of the plurality of training samples:
processing, using the blood pressure prediction model, the corresponding training single-lead ECG data to generate a corresponding predicted blood pressure; and
determining a corresponding loss based on the corresponding predicted blood pressure and the corresponding ground-truth blood pressure; and
updating parameters of the blood pressure prediction model based on the corresponding losses.

14. The system of claim 13, wherein obtaining the single-lead ECG data comprises obtaining the single-lead ECG data using a single-lead sensor affixed to the individual.

15. The system of claim 13, wherein obtaining the single-lead ECG data comprises obtaining the single-lead ECG data using a single-lead sensor integrated by a wearable device worn by the individual.

16. The system of claim 13, wherein the CNN model architecture comprises:
an input layer;
one or more one-dimensional convolution layers;
a max pooling layer;
a flattening layer; and
one or more densely-connected layers.

17. The system of claim 16, wherein:
a first layer of the one or more one-dimensional convolution layers comprises a first plurality of filters, a first rectified linear unit (ReLU) activation function, and a first kernel size; and
a second layer of the one or more one-dimensional convolution layers comprises a second plurality of filters, a second ReLU activation function, and a second kernel size different from the first kernel size.

18. The system of claim 16, wherein the max pooling layer comprises a pool size of 2.

19. The system of claim 16, wherein a first layer of the one or more densely-connected layers comprises a plurality of nodes and a rectified linear unit (ReLU) activation function.

20. The system of claim 13, wherein the single-lead ECG data is obtained by pre-processing raw single-lead ECG signals acquired by a single-lead sensor using one or more filters.

21. The system of claim 20, wherein the one or more filters are configured to at least one of:
attenuate noise from a power supply;
attenuate high-frequency noise from muscle artifacts; or
attenuate low-frequency noise due to baseline wander or breathing motions.

22. The system of claim 20, wherein the one or more filters comprise:
a notch filter to attenuate noise at 50 Hertz (Hz) or 60 Hz;
a high-pass filter with a cut-off frequency of 0.05 Hz; and
a low-pass filter with a cut-off frequency of 150 Hz.

23. The system of claim 13, wherein processing, using the blood pressure prediction model having the CNN model architecture, the single-lead ECG data to predict the blood pressure of the individual comprises processing, using the blood pressure prediction model having the CNN model architecture, the single-lead ECG data to predict a systolic blood pressure (SBP) value and a diastolic blood pressure (DBP) value of the individual.

24. The system of claim 23, wherein:
the corresponding predicted blood pressure generated during the training process comprises a corresponding predicted SBP value and a corresponding predicted DBP value;
the corresponding ground-truth blood pressure of each training sample comprises a corresponding ground-truth SBP value and a corresponding ground-truth DBP value; and
determining the corresponding loss comprises:
determining a corresponding SBP loss based on the corresponding predicted SBP value and the corresponding ground-truth SBP value; and
determining a corresponding DBP loss based on the corresponding predicted DBP value and the corresponding ground-truth DBP value.
